(19) United States
(12) Patent Application Publication
(10) Pub. No.: US 2010/0235994 A1
(43) Pub. Date: Sep. 23, 2010

(54) MODULAR OPERATING TABLE COVER

(76) Inventors: Rainhard Wedeleit, Hamburg (DE); Martin Deli, Mulheim (DE); Martin Busch, Witten (DE); Dietrich H.W. Grönemeyer, Sprockhovel (DE)

Correspondence Address: BERLINER & ASSOCIATES, 555 WEST FIFTH STREET, 31ST FLOOR, LOS ANGELES, CA, 90013, US

(21) Appl. No.: 12/601,599

(22) PCT Filed: Oct. 25, 2007
(86) PCT No.: PCT/EP2007/009245

§ 371 Date: Jun. 8, 2010

(30) Foreign Application Priority Data
Oct. 26, 2006 (DE) 10 2006 050 564.6

(51) Int. Cl. A61G 13/12; A61B 6/04; A61G 13/08

(52) U.S. Cl. 5/601

(57) ABSTRACT

The invention relates to an operating table top (4) consisting of one or several basic sections (5) and one or several modules (6), with such modules (6) and basic sections (5) being adjustable relative to each other and such modules (6) being designed so as to be separable from the operating table top (4), and with such modules (6) being designed so as to be replaceable with additional supporting surfaces (8) separably connected to the basic sections (5), and with the operating table top (4) and its attached additional supporting surfaces (8) being of suitable design so that they can be introduced into radiographic equipment, magnetic resonance imaging and/or computer tomography systems and radiographic, magnetic resonance or computer tomographic images can be produced. Moreover, the invention relates to a pertinent operating table system as well as a pertinent operating table (1). By means of the operating table system according to the invention it is possible to examine a patient, for example by magnetic resonance or computer tomography, without the necessity of moving them off the operating table (1).

The invention relates to an operating table top consisting of one or several basic sections and one or several additional modules with such modules and basic sections being adjustable relative to each other and the modules being designed so as to be separable from the operating table top.

Operating tables provided with a variety of adjusting features usually actuated by electric motor are basically known. EP 0 917 687 A1, for example, proposes an operating table top comprising a basic section which is connected to a supporting column as well as several additional table top sections attached to the basic section so as to be tiltable, primarily around a horizontal tilting or pivoting axis. In this manner the patient resting on the operating table top can be brought into a position most favorable for the envisaged operation. If necessary, additional modules which are also adjustable may be attached to such an operating table top. This enables operating tables to be suitably extended as and when required.

However, a drawback associated with such operating table tops known from prior art is that to a large extent these supporting structures are unsuited for use with X-ray equipment, magnetic resonance imaging or computer tomography systems. Especially the great variety of adjusting features offered by customary operating tables makes it necessary that a relatively great number of metal parts have to be integrated into the operating table top. It is known, however, that metal objects positioned inside a magnetic resonance imaging system are unwanted because they may distort the magnetic field and cause current induction in such metal objects. Moreover, in radiography and computer tomographic imaging as well metals are detrimental as they are entirely or to some degree opaque to X-rays and in particular produce streak artifacts.

Due to the fact that the operating table top itself cannot be positioned inside the relevant imaging system the patient therefore must be transferred from the operating table and put onto another support to enable the examination to be carried out. On the one hand, this is not only time consuming and takes considerable effort but on the other may even be hazardous for the patient depending on injury or disease the patient suffers from. Moreover, in case of seriously injured persons any transfer or repositioning may be extremely painful to the patient.

The drawbacks described above will be especially pronounced if a patient has to be transferred several times: for example, if a magnetic resonance or computer tomographic image has to be produced after or during an operation to enable the operation either to be continued based on the results thus obtained or to perform another operation. In this case the patient has to be moved at least twice, that is from the operating table to the supporting base suited for the tomographic examination and then back onto the operating table.

Proceeding from the state of the art as described hereinbefore, it is thus the objective of the present invention to propose an operating table top that may serve both as an operating table cover offering several adjusting features and as a cover or top capable of being moved into a radiographic or tomographic imaging system or device.

According to the invention this objective is reached by providing an operating table top consisting of one or several basic sections and one or several modules, with such modules and basic sections being adjustable relative to each other and such modules being designed so as to be separable from the operating table top and, furthermore, such modules being designed so as to be replaceable with additional supporting surfaces separably connected to the basic sections, and with the operating table top and its attached additional supporting surfaces being of suitable design so that they can be introduced into radiographic equipment, magnetic resonance imaging and/or computer tomography systems and radiographic, magnetic resonance or computer tomographic images can be produced. Moreover, the invention relates to an operating table system comprising the operating table top described herein and the additional supporting surfaces referred to, as well as an operating table consisting of an operating table top and a supporting column which carries the operating table top attached to it.

Core idea of the invention is to use an operating table top to which the modules, arranged on the basic sections of the operating table top and ensuring the variety of adjusting features of the entire operating table top to be achieved, are attached in such a manner that they can be removed and re-mounted without difficulty. To be able to use the operating table top for imaging purposes the modules will be replaced with add-on covering surfaces on which a patient, and in particular his or her extremities, will be positioned. Although there are as a rule no or only minor adjusting possibilities for the add-on covering surfaces, said covering surfaces—other than the modules—offer advantages in that they are compatible with radiography, magnetic resonance and/or computer tomography technologies. What is more, when the process of producing the desired images has been completed these add-on covering surfaces may also be easily removed and replaced with the modules so that a fully functioning operating table is available again.

As a rule, the operating table top will consist of one or several basic sections on which the trunk of a patient, i.e. roughly from shoulders to fundament, will be positioned. The adjustable modules which are coupled to the basic section primarily serve as supporting elements for head, arms and legs. If such an adjustable module is replaced with an add-on supporting surface all that is needed is to briefly support for example a patient's arm or leg until the add-on supporting surface has been coupled to the basic section. The trunk of the patient in this case remains in position on the basic section or sections so that it is no longer necessary to remove the relevant patient from the table.

As mentioned above, the add-on supporting surfaces must be suited for production of radiographic, magnetic resonance or computer tomographic images. Depending on the respective application of the operating table top it will be sufficient for the add-on supporting surfaces to be suitable for use with one of these device types; however, it is considered expedient if they are suited for all the systems named here. The same applies basically to other examination devices and systems.

Especially for an operating table top to be used inside a magnetic resonance tomograph with such add-on supporting surfaces attached it is considered expedient that the add-on supporting surfaces and/or the basic sections which remain attached to the operating table top be manufactured of a diamagnetic material or a slightly paramagnetic material which leaves the produced magnetic field undistorted or, at the most, only insignificantly distorted. The magnetic susceptibility constant Xₘ should equal that of the examined tissue, i.e. should be near zero. The susceptibility constant should at least be <100, preferably <10. Therefore, the use of ferromagnetic materials the susceptibility constant of which is significantly higher, for example steel, is always to be avoided.

Another undesirable design characteristic in connection with magnetic resonance imaging constitute ring-shaped metal structures which may also cause the induction of currents resulting in a distortion of the magnetic field. Accordingly, neither the add-on supporting surfaces nor the basic sections should contain such ring-shaped metal structures. Ideally, add-on supporting surfaces and/or basic sections should be manufactured without metal components being used to the extent feasible. Integrating metal components into operating table tops to be used in X-ray equipment or computer tomographs is rather undesirable as well because metals are entirely or to a varying degree opaque to X-rays. To the extent feasible materials should be used that are transparent to X-rays.

Materials such as plastic, foamed plastic, rubber, textiles or wood are particularly suited for the add-on supporting surfaces and basic sections, with special attention being required, however, to make sure the materials put to use in fact are compatible with the envisaged magnetic resonance and/or radiographic techniques. Appropriate materials are basically known to persons skilled in the art.

For example, the add-on supporting surfaces may be secured to the basic sections by means of rails, pegs and recesses provided to accommodate the pegs, or by means of a tongue and groove system. Other conceivable connecting means are hooks meant to interact with specially adapted eyelets while Velcro strips or zip fasteners may also be used. Notably, the suitability of these means of connection between the add-on supporting surfaces and the basic sections with respect to magnetic resonance or radiographic examination techniques must of course also be ensured. Accordingly, rails designed to be pushed into each other as well as interacting pegs and recesses should possibly not be manufactured of metal but, for example, of an appropriate plastic material. Moreover, the connection should be designed so as to be established expeditiously to enable the modules to be quickly replaced with the add-on supporting surfaces. This will make working easier for the medical staff and be more comfortable for the patient. After all, in many instances the time factor is of great significance when treating patients.

Aside from the operating table top or the operating table system comprising the operating table top and add-on supporting surfaces the invention also relates to an operating table that consists of an operating table top as described hereinbefore and a supporting column to which the operating table top is attached. Usually, the supporting column is directly connected with the basic section. Moreover, the supporting column may be provided with control elements, an electric drive system to adjust the position of the operating table, a power source and similar features. In this case an electrical connection to the adjustable modules has to be established via cable, contacting faces or similar means. The operating table is frequently controlled with the help of a remote.

To enable the operating table top to be separately moved into an X-ray apparatus or tomograph it is considered expedient for the operating table top to be designed so as to be removable from the supporting column. Any electrical connections that may be provided between the supporting column and operating table top should be capable of being easily detached and established again, for example by arranging appropriate contact faces in the components. Basically, the operating table may also be adjusted mechanically. However, especially preferred is an electrical adjustment of the modules relative to the basic sections, possibly inside the modules or basic sections as well. In this case, the adjustment can be brought about more quickly and without difficulty.

Further elucidation of the invention is provided through the enclosed figures, where

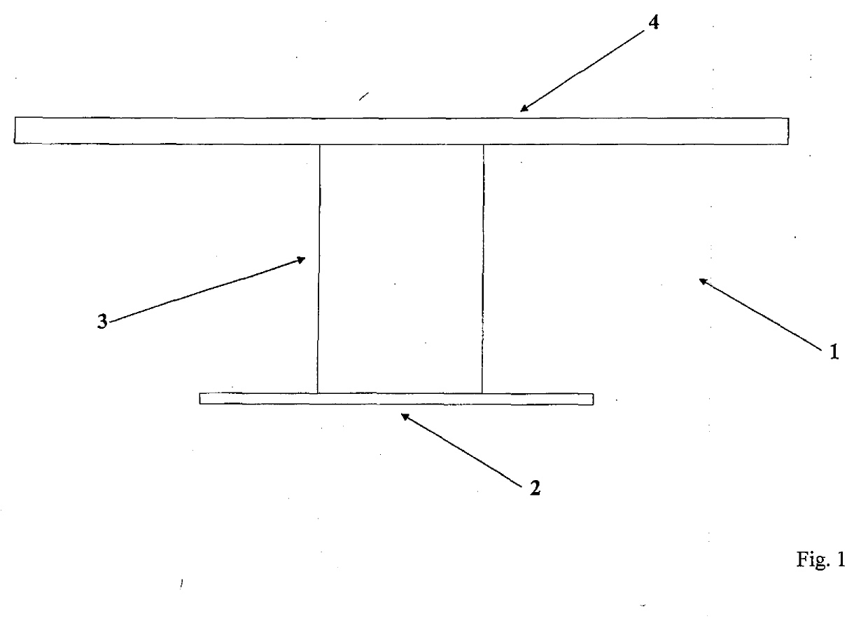

FIG. 1 is a schematic representation of an operating table in accordance with the invention as side view;

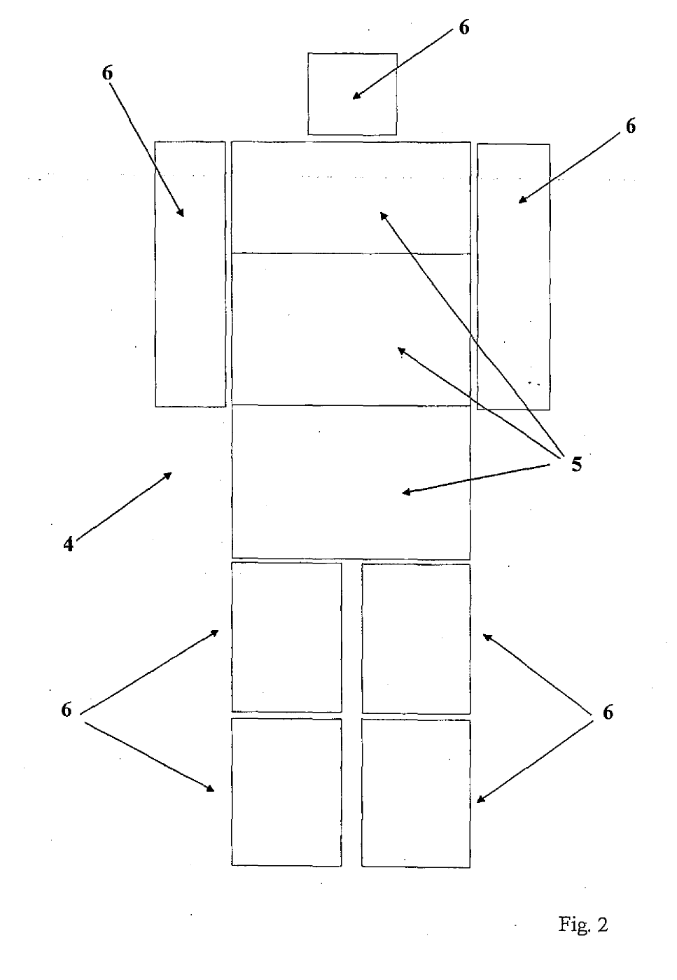

FIG. 2 is a top view of the inventive operating table top;

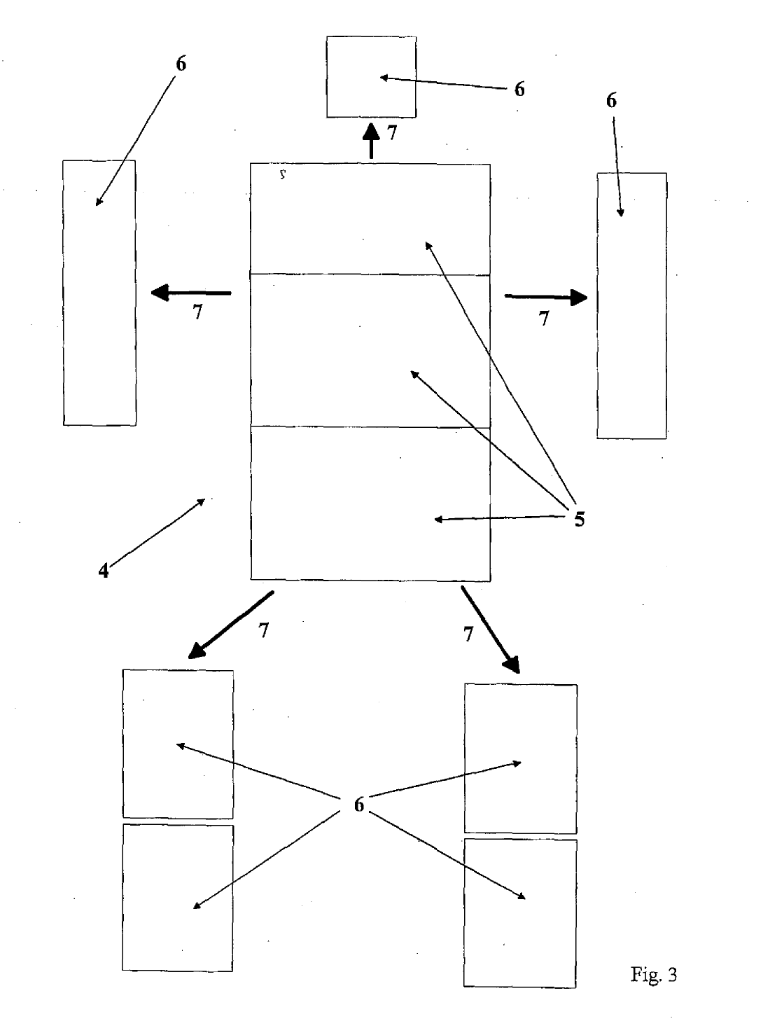

FIG. 3 is a top view showing how the adjustable modules are removed from the operating table top;

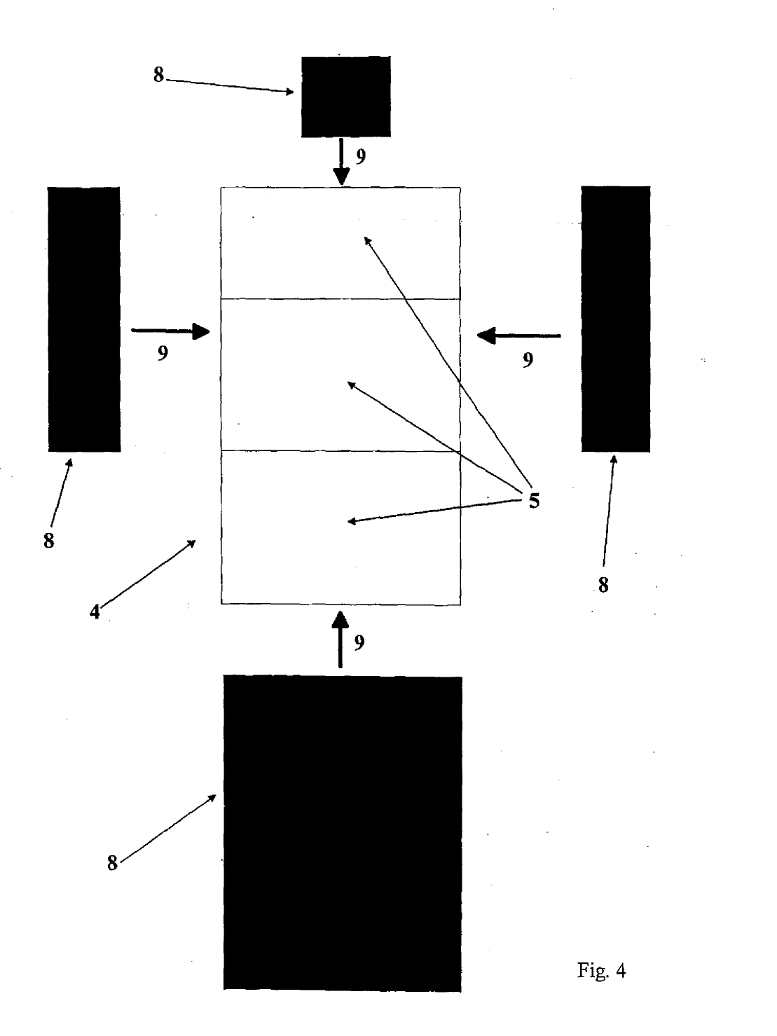

FIG. 4 is a top view showing how the add-on supporting surfaces are mounted on the inventive operating table top, and

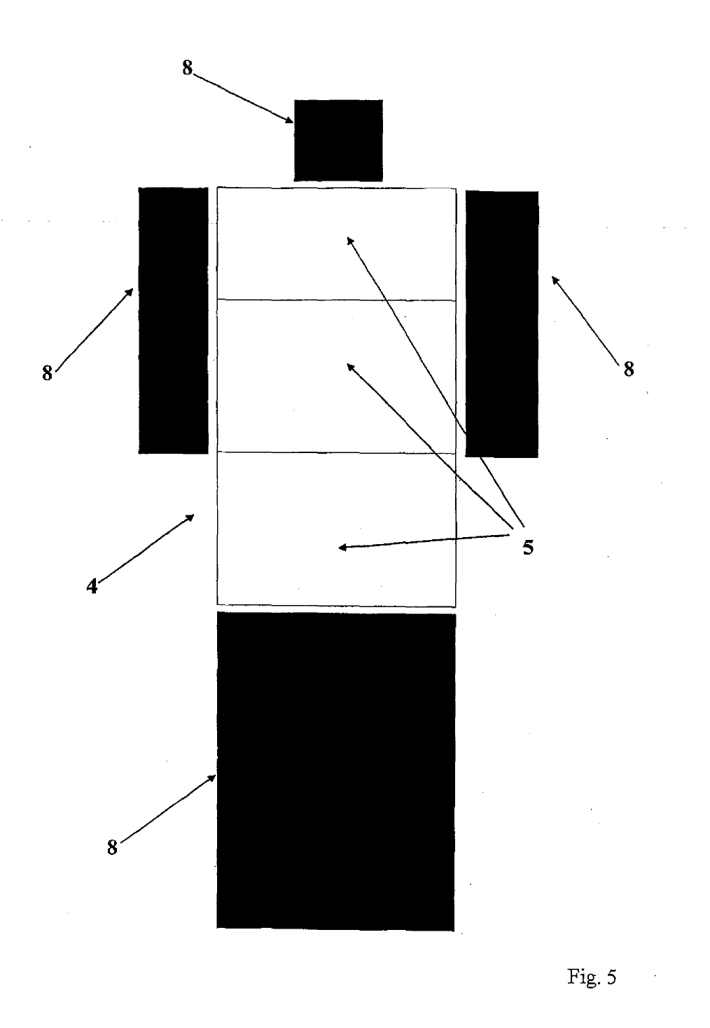

FIG. 5 is a top view of the inventive operating table top with the add-on supporting surfaces attached.

In FIG. 1 an operating table 1 is shown according to the invention which consists of a baseplate 2, a supporting column 3 and an operating table top 4. While the patient rests on the operating table top 4 the supporting column accommodates among other items the operating and control elements.

FIG. 2 is a top view of the operating table top 4 proposed by the present invention. The operating table top 4 comprises three basic sections 5 intended to accommodate the patient's shoulders, back and fundament. Moreover, modules 6 are attached to the basic sections 5 serving to support the head, arms as well as legs. The modules 6 have been provided with a number of adjusting features relative to the basic sections 5a or also within the modules 6 themselves.

In FIG. 3 it is shown how the modules 6 can be removed from the basic sections 5 of the operating table top 4. For this purpose the modules 6 are moved away from the basic sections 5 in the manner indicated by arrows 7. The method of connecting the modules 6 with the basic sections 5 should make sure that both the attachment and the removal of modules 6 can be brought about as quickly as possible. Electrical connections may, for example, be established by providing appropriate contact faces. After the modules 6 have been removed the add-on supporting surfaces 8 can be attached to the operating table top 4 as depicted schematically in FIG. 4 in the direction indicated by arrows 9. For example, the connection or attachment may be achieved or established by means of interacting rails, interacting pegs and recesses or via a tongue and groove system. Other than the modules 6 which offer a variety of adjusting features the add-on supporting surfaces 8 are usually of rigid construction.

FIG. 5 finally shows the operating table top 4 configured in such a manner that it can be moved into an X-ray apparatus or a magnetic resonance or computer tomographic imaging system. The modules 6 have been completely replaced with add-on supporting surfaces 8. In this configuration the operating table top is compatible with magnetic resonance and/or X-ray examination equipment. After the desired images have been produced, for instance as preparation for or to continue an operation, the original state can be restored by replacing the add-on supporting surfaces 8 with the modules 6.

1. Operating table top consisting of one or several basic sections (5) and one or several modules (6), with such modules (6) and basic sections (5) being adjustable relative to each other and such modules (6) being designed so as to be separable from the operating table top (4), characterized in that such modules (6) being designed so as to be replaceable with additional supporting surfaces (8) separably connected to the basic sections (5), and with the operating table top (4) and its attached additional supporting surfaces (8) being of suitable design so that they can be introduced into radiographic equipment, magnetic resonance imaging and/or computer tomography systems and radiographic, magnetic resonance or computer tomographic images can be produced.

2. Operating table system comprising an operating table top (4) according to claim 1 and add-on supporting surfaces (8) separably connectable to the basic sections (5) of the operating table top (4), with the operating table top (4) with attached add-on supporting surfaces (8) being of suitable design so that they can be introduced into radiographic equipment, magnetic resonance imaging and/or computer tomography systems and radiographic, magnetic resonance or computer tomographic images can be produced.

3. Operating table system according to claim 2, characterized in that the add-on supporting surfaces (8) and/or the basic sections (5) are manufactured of a material having a magnetic susceptibility of <100.

4. Operating table system according to claim 2, characterized in that the add-on supporting surfaces (8) and/or the basic sections (5) do not contain ferromagnetic substances.

5. Operating table system according to claim 2, characterized in that the add-on supporting surfaces (8) and/or the basic sections (5) do not contain ring-shaped metal structures.

6. Operating table system according to claim 2 characterized in that the add-on supporting surfaces (8) and/or the basic sections (5) do not contain metals.

7. Operating table system according to claim 2, characterized in that the add-on supporting surfaces (8) and/or the basic sections (5) are manufactured of a material transparent to X-rays.

8. Operating table system according to claim 2, characterized in that the add-on supporting surfaces (8) and/or the basic sections (5) are manufactured of plastic, foamed plastic, textile materials, rubber or wood.

9. Operating table system according to claim 2, characterized in that the add-on supporting surfaces (8) are secured to the basic sections (5) by means of rails, pegs and recesses designed to accommodate such pegs or via a tongue and groove system.

10. Operating table system according to claim 2, characterized in that the add-on supporting surfaces (8) are secured to the basic sections (5) by means of hooks and eyelets designed for a connection to such hooks.

11. Operating table system according to claim 2, characterized in that the add-on supporting surfaces (8) are secured to the basic sections (5) by means of Velcro strips or zip fasteners.

12. Operating table consisting of an operating table top (4) according to claim 1 and a supporting column (3) on which the operating table top (4) is secured.

13. Operating table according to claim 12, characterized in that the operating table top (4) is designed so as to be removable from the supporting column (3).

14. Operating table according to claim 12, characterized by electrical adjusting features of the modules (6) to enable adjustments to be made relative to the basic sections (5).
